(19) United States
(12) Patent Application Publication
(10) Pub. No.: US 2020/0371086 A1
(43) Pub. Date: Nov. 26, 2020

(54) QUALITY CONTROL SUBSTANCES FOR USE WITH MICROSCOPY-BASED URINE SEDIMENT ANALYZERS AND METHODS OF USING THE SAME

(71) Applicant: Siemens Healthcare Diagnostics Inc., Tarrytown, NY (US)

(72) Inventor: Kausik Das, Quincy, MA (US)

(73) Assignee: Siemens Healthcare Diagnostics Inc., Tarrytown, NY (US)

(21) Appl. No.: 16/640,590

(22) PCT Filed: Aug. 24, 2018
(86) PCT No.: PCT/US2018/047874

§ 371 Date: Feb. 20, 2020

Related U.S. Application Data
(60) Provisional application No. 62/550,852, filed on Aug. 28, 2017.
(60) Provisional application No. 62/608,656, filed on Dec. 21, 2017.

(51) Int. Cl. G01N 33/493; G01N 33/96

(52) U.S. Cl. CPC G01N 33/493; G01N 33/96; G01N 2800/7028; G01N 2333/405; G01N 2333/39

(57) ABSTRACT

Provided herein are quality control substances for use with a microscopy-based urine sediment analyzer. The quality control substances comprise a urine matrix and a cancer cell, an algae cell, a yeast cell, egg white, or any combination thereof. Methods of detecting the presence of an analyte in a urine sample from a subject, quality control substances for use in identifying the presence of one or more analytes in a urine sample from a subject, and the use of a cancer cell, an algae cell, a yeast cell, egg white, or any combination thereof in the manufacture of a quality control substance for use with a microscopy-based urine analyzer are also provided.

### CROSS REFERENCE TO RELATED APPLICATIONS

This application claims priority to U.S. Provisional Application No. 62/550,852, filed Aug. 28, 2017, and U.S. Provisional Application No. 62/608,656, filed Dec. 21, 2017, the disclosure of each of which is hereby incorporated by reference in its entirety.

### FIELD OF THE INVENTION

Provided herein are quality control substances for use with microscopy-based urine sediment analyzers and methods of using the same.

### BACKGROUND

Microscopy-based urine sediment analyzers evaluate urine samples for the presence of various analytes based on the morphologies of those analytes. To ensure that the sediment analyzers are properly detecting analytes based upon their morphologies, quality controls are required. The current sediment urinalysis quality control (QC) materials, however, do not serve as adequate controls for all common analytes.

### SUMMARY OF THE INVENTION

Provided herein are quality control substances for use with a microscopy-based urine sediment analyzer, the quality control substance comprising a urine matrix and a cancer cell, an algae cell, a yeast cell, egg white, or any combination thereof.

Methods of detecting the presence of an analyte in a urine sample from a subject are also disclosed herein. The methods comprise analyzing any of the quality control substances disclosed herein with a microscopy-based urine sediment analyzer to determine a morphology of components within the quality control substance and comparing the morphology of the components within the quality control substance to a morphology of analytes within the urine sample, wherein a matching morphology between the analyte and the quality control substance indicates the presence of the analyte in the urine sample.

Also provided are quality control substances for use in identifying the presence of one or more analytes in a urine sample from a subject and the use of a cancer cell, an algae cell, a yeast cell, egg white, or any combination thereof in the manufacture of a quality control substance for use with a microscopy-based urine analyzer.

### BRIEF DESCRIPTION OF THE DRAWINGS

The summary, as well as the following detailed description, is further understood when read in conjunction with the appended drawings. For the purpose of illustrating the disclosed quality control substances and methods, there are shown in the drawings exemplary embodiments of the quality control substances and methods; however, the quality control substances and methods are not limited to the specific embodiments disclosed. In the drawings:

FIG. 1 is a representative Urised2 image of a sample with targeted RBC concentration of 500/μL.

FIG. 2 is a representative image of WBC and NEC positive urine samples prepared by adding a hybridoma cell suspension in urine.

FIG. 3 is a representative Urised2 image of a urine sample with a NEC concentration of 82.28/μL. The image shows the presence of round shaped cells with defined smooth edges.

FIG. 4 is a representative Urised2 image of a urine sample with a YEA concentration of 18.48/μL. The image shows the presence of round-oval shaped single and budding yeast cells.

FIG. 5 is a representative Atellica UAS 800 image of a urine sample with a YEA concentration of 80-100/μL. The image shows the presence of round-oval shaped single and budding yeast cells.

FIG. 6 is a representative Atellica UAS 800 image of a urine sample with a YEA concentration of 652-674/μL. The image shows the presence of round-oval shaped single and budding yeast cells.

FIG. 7 is a representative Atellica UAS 800 image of a MUC positive urine sample.

FIG. 8 is a representative Urised2 image indicating the presence of PAT in urine.

### DETAILED DESCRIPTION OF ILLUSTRATIVE EMBODIMENTS

The disclosed quality control substances and methods may be understood more readily by reference to the following detailed description taken in connection with the accompanying figures, which form a part of this disclosure. It is to be understood that the disclosed quality control substances and methods are not limited to the specific quality control substances and methods described and/or shown herein, and that the terminology used herein is for the purpose of describing particular embodiments by way of example only and is not intended to be limiting of the claimed quality control substances and methods.

Unless specifically stated otherwise, any description as to a possible mechanism or mode of action or reason for improvement is meant to be illustrative only, and the disclosed quality control substances and methods are not to be constrained by the correctness or incorrectness of any such suggested mechanism or mode of action or reason for improvement.

Throughout this text, the descriptions refer to quality control substances and methods of using said quality control substances. Where the disclosure describes or claims a feature or embodiment associated with a quality control substance, such a feature or embodiment is equally applicable to the methods of using said quality control substance. Likewise, where the disclosure describes or claims a feature or embodiment associated with a method of using a quality control substance, such a feature or embodiment is equally applicable to the quality control substance.

It is to be appreciated that certain features of the disclosed quality control substances and methods which are, for clarity, described herein in the context of separate embodiments, may also be provided in combination in a single embodiment. Conversely, various features of the disclosed quality control substances and methods that are, for brevity, described in the context of a single embodiment, may also be provided separately or in any subcombination.

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily referring to the same embodiment.

Unless expressly stated to the contrary, “or” refers to an inclusive “or” and not to an exclusive “or.” For example, a condition A or B is satisfied by anyone of the following: A is true (or present) and B is false (or not present), A is false (or not present) and B is true (or present), and both A and B are true (or present). An inclusive “or” may be understood as being the equivalent to: at least one of condition A or B.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

The term “negative urine” refers to a urine specimen that exhibits negative results with sediment analysis.

As used herein, the term “urine matrix” refers to urine that is stabilized with an anti-microbial agent such as, for example, with the use of a BD Vacutainer® UA Preservative Tube (BD Diagnostics, Catalog #364992). The difference between native and preserved urine is the presence of a preservative chemical in preserved urine.

The following abbreviations are used throughout the disclosure: bacteria (BAC); crystal (CRY); non-squamous epithelial cells (NEC); pathological cast (PAT); quality control (QC); red blood cell (RBC); urinalysis (UA); white blood cell (WBC); yeast (YEA).

Current commercial QC materials have only some of the sediment analytes found in urine (such as red blood cells (RBC), white blood cells (WBC), and crystal (CRY)). These QC materials, however, do not contain the controls to analyze bacteria (BAC), yeast (YEA), pathological cast (PAT), or non-squamous epithelial cells (NEC). Because the presence of BAC, YEA, PAT, and/or NEC, even in small quantities, can be indicative of pathological conditions, it is important to check that the analyzer is properly detecting these analytes. The instant disclosure provides quality control substances for detection of these analytes.

Provided herein are quality control substances for use with a microscopy-based urine sediment analyzer, the quality control substance comprising:

a urine matrix; and

a cancer cell, an algae cell, a yeast cell, egg white, or any combination thereof.

Cancer cells can be included in the quality control substance to serve as a morphological control for non-squamous epithelial cells present in a urine sample. Suitable cancers cells will have a similar morphology to the non-squamous epithelial cells including a round shape with smooth and defined perimeters, a size in the range of 30-40 micrometer, and granulated cytoplasm and a dark-round nucleus. Suitable cancer cells include SKBR-3 cells or H-1975 cells. SKBR-3 cells are human breast cancer cells and can include ATCC® HTB-30® cells. H-1975 cells are human lung cancer cells and can include ATCC® CRL-5908™ cells. In some embodiments, the quality control substance comprises SKBR-3 cells. In some embodiments, the quality control substance comprises H-1975 cells. In some embodiments, the quality control substance comprises both SKBR-3 and H-1975 cells. SKBR-3 cells can be grown in McCoy's 5A medium with 10% FBS (Fetal bovine serum) and H-1975 cells can be grown in DMEM (Dulbecco's Modified Eagle's Medium). After collecting a sufficient number of cells, the cells can be washed with PBS (phosphate buffer saline) to remove the growth media. The H-1975 cells can then be fixed by incubating the cells at 2-8° C. for 24 hours in 2% formaldehyde in PBS. SKBR-3 cells can be fixed by incubating the cells at 2-8° C. for 24 hours in 2% formaldehyde in PBS. Cells obtained by the above mentioned methods and fixed with glutaraldehyde, or other common cell preservatives (including diazolidinyl urea or imidazolidinyl urea) can be used as a morphological control for non-squamous epithelial cells.

Algae cells can be included in the quality control substance to serve as a morphological control for pathological cast present in a urine sample. Suitable algae cells have a similar morphology to the pathological cast including a cylindrical shape and a granulated interior. Suitable algae cells can comprise, for example, diatom (such as that from Nile Biological Inc.). Diatoms are eukaryotic algae, which, under microscope, appear to have a two parallel edges with granular interior. Such distinct morphological aspect mimics the cylindrical shape and granularity of pathological casts. The diatom algae can be obtained in water suspension. The suspension can be mixed by inverting the container and added (approximately 0.5 mL) to 3 mL negative urine.

Yeast cells can be included in the quality control substance to serve as a morphological control for yeast present in a urine sample. Exemplary yeast include a naturally occurring yeast present in urine or Saccharomyces cerevisiae. In some embodiments, yeast cells (Candida albican) can be isolated from the urine of a control subject (i.e. not the subject whose urine is being analyzed) and added to the quality control substance. In some embodiments, Saccharomyces cerevisiae can be added to the quality control substance. Yeast cells grown in urine medium containing a sucrose solution are correctly recognized as yeast by the sediment analyzers. The yeast cells grown in urine or PBS without sucrose solution, however, are both correctly recognized as yeast and also incorrectly recognized as RBC by the sediment analyzers. (Table 7). Thus, in some embodiments, the yeast cells are present in a solution comprising a sucrose solution or are obtained from a sucrose solution.

Egg white can be included in the quality control substance to serve as morphological control for mucus in urine sample. Egg white is a protein solution with a small quantity of carbohydrate and sodium salt. The egg white mucoid material (approximately 1 mL) can be added into negative urine (3 mL) to create a sample with a positive mucus result.

In some embodiments, the quality control substance can comprise a cancer cell and an algae cell. In some embodiments, the quality control substance can comprise a cancer cell and a yeast cell. In some embodiments, the quality control substance can comprise an algae cell and a yeast cell. In some embodiments, the quality control substance can comprise a cancer cell and egg white. In some embodiments, the quality control substance can comprise an algae cell and egg white. In some embodiments, the quality control substance can comprise a yeast cell and egg white. In some embodiments, the quality control substance can comprise a cancer cell, an algae cell, and egg white. In some embodiments, the quality control substance can comprise an algae cell, a yeast cell, and egg white. In some embodiments, the quality control substance can comprise a cancer cell, an algae cell, and a yeast cell. In some embodiments, the quality control substance can comprise a cancer cell, an algae cell, a yeast cell, and egg white.

The disclosed quality control substances can further comprise a crystal, a bacterial cell, a sperm cell, a white blood cell, a red blood cell, a hyaline cast, or any combination thereof.

Also disclosed are methods of detecting the presence of an analyte in a urine sample from a subject. The disclosed methods comprise analyzing any of the herein disclosed quality control substances with a microscopy-based urine sediment analyzer to determine a morphology of components within the quality control substance and comparing the morphology of the components within the quality control substance to a morphology of analytes within the urine sample, wherein a matching morphology between the analyte and the quality control substance indicates the presence of the analyte in the urine sample.

The disclosed methods can be used to detect the presence of non-squamous epithelial cells within the urine sample. In embodiments wherein the analyte is a non-squamous epithelial cell, the quality control substance can comprise a cancer cell.

The disclosed methods can be used to detect the presence of pathological cast within the urine sample. In embodiments wherein the analyte is a pathological cast, the quality control substance can comprise an algae cell.

The disclosed methods can be used to detect the presence of yeast within the urine sample. In embodiments wherein the analyte is a yeast cell, the quality control substance can comprise a yeast cell.

The disclosed methods can be used to detect the presence of mucus within the urine sample. In embodiments wherein the analyte is mucus, the quality control substance can comprise egg white.

The disclosed methods can be used to detect the presence of: non-squamous epithelial cells and pathological cast; non-squamous epithelial cells and yeast; pathological cast and yeast; non-squamous epithelial cells and mucus; pathological cast and mucus; yeast and mucus; non-squamous epithelial cells, pathological cast, and yeast; non-squamous epithelial cells, pathological cast, and mucus; non-squamous epithelial cells, yeast and mucus; pathological cast, yeast, and mucus; or non-squamous epithelial cells, pathological cast, yeast, and mucus. In embodiments wherein the methods are used to detect a combination of the above analytes, the quality control substances can have a combination of cancer cells (such as SKBR-3 cell or an H-1975 cell), algae cells (such as diatom), yeast cells (such as Candida albican or Saccharomyces cerevisiae), and/or egg whites.

Also provided are quality control substances for use in identifying the presence of one or more analytes in a urine sample from a subject.

The use of a cancer cell, an algae cell, a yeast cell, egg white, or any combination thereof in the manufacture of a quality control substance for use with a microscopy-based urine analyzer are provided herein.

### Examples

The following examples are provided to further describe some of the embodiments disclosed herein. The examples are intended to illustrate, not to limit, the disclosed embodiments.

### Preparation of RBC Positive Urine

RBC positive urine was prepared as follows:
1) Human blood (7-8 ml) was drawn into 10 mL-Heparin tube and stored at room temperature for 2 days, resulting in the stabilization of the blood cells.
2) 10 μL aliquots of the stabilized blood were separately added into three different media (10 mL) as shown in Table 1. Aliquots of 3 mL from each preparation were analyzed on Urised2.

TABLE 1
Different preparations of stabilized RBC contrived urine samples

| | Blood | Urised2 |
|---|---|---|
| Medium | samples added | Results |
| 10 mL of | 10 μL of heparin | RBC = 6438.96 |
| Negative Urine | stabilized blood | |
| 10 mL of saline | 10 μL of heparin | RBC = 6211.04 |
| (0.9% NaCl) | stabilized blood | |
| 10 mL of PBS | 10 μL of heparin | RBC = 6182.0 |
| (phosphate buffer Saline) | stabilized blood | |

3) From the above stocks, diluted samples were made targeting 3000/μL and 500/μL RBC using respective media as diluent.

TABLE 2
Preparation of diluted samples to
obtain targeted RBC concentration

| | | Target RBC | Observed Urised2 |
|---|---|---|---|
| Stock volume | Blank media | concentrations | Result |
| 1.16 mL | 1.13 mL | 3000/μL | RBC = 2944.48/μL |
| Urine stock | negative urine | | |
| 1.21 mL | 1.29 mL | 3000/μL | RBC = 1546.16/μL |
| Saline stock | Saline | | |
| 1.21 mL | 1.29 mL | 3000/μL | RBC = 1684.32/μL |
| PBS stock | PBS | | |
| 0.194 mL | 2.31 mL | 500/μL | RBC = 531.52/μL |
| Urine stock | negative urine | | |

4) Similar dilutions were repeated with urine based stock and negative urine with 2 day old RBC (2 day old whole blood in Heparin tubes), as shown in Table 3, to achieve RBC concentrations of 1000, 800, 400, 100 and 50/μL. A stock was prepared by adding 10 μL of 2-day old whole blood in 10 mL of negative urine. RBC concentration of the stock was 6875.44/μL.

TABLE 3
Preparation of linearity samples with targeted RBC concentrations

| | | | Observed |
|---|---|---|---|
| Urine | Negative | Target RBC | Urised2 RBC |
| based stock | Urine volume | concentration | result |
| 0.364 mL | 2.136 mL | 1000/μL | 1063.92/μL |
| 0.291 mL | 2.209 mL | 800/μL | 910.80/μL |
| 0.145 mL | 2.355 mL | 400/μL | 352.00/μL |
| 0.036 mL | 2.464 mL | 100/μL | 105.60/μL |
| 0.018 mL | 2.482 mL | 50/μL | 48.40/μL |

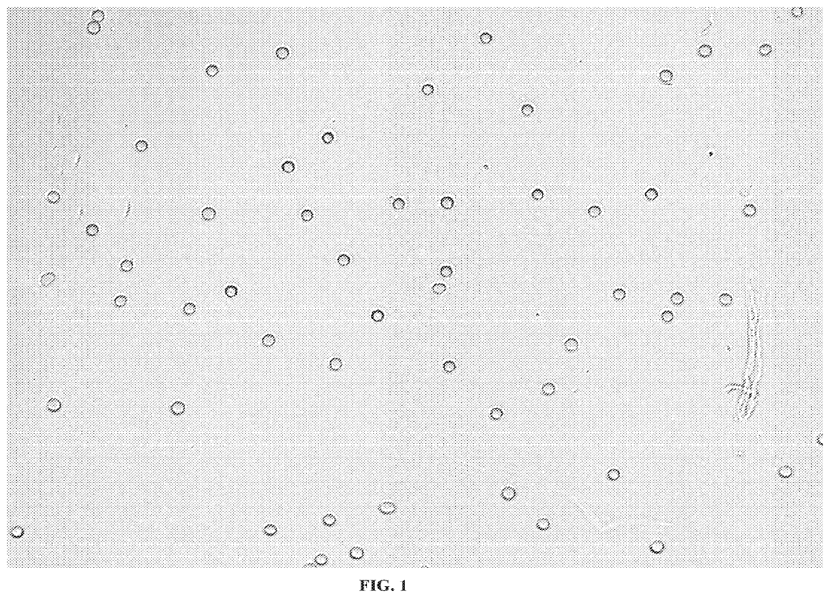

A representative Urised2 image of the sample with targeted RBC concentration 500/μL is shown in FIG. 1.

### Preparation of NEC and WBC Positive Urine Using Hybridoma Cells

NEC and WBC positive urine samples were created as follows:
1) Hybridoma cells (hybridization between mouse spleen cells and myeloma cells (p653 cells)) were grown internally within Siemens Healthineers laboratory following internal procedure. Cells were obtained in growing medium. Cells were added into 10 mL of culture medium, centrifuged (200-500 g at 5-7 minutes) to remove the storage medium, resuspended into the culture medium, and placed the container in CO₂-incubator. Once the sufficient cells were grown, the medium was removed by centrifugation and the cells were harvested in PBS.
2) Cells were then fixed with 2% Formaldehyde in MDM medium for 24 hour at 2-8° C. Formaldehyde solution was removed by centrifugation and the fixed cells were resuspended into MDM medium.
3) 0.5 mL of cell suspension (conc.=˜500/μL) was added in 5 mL of negative urine. The suspension was homogenized and the sample was analyzed on Urised2, as shown in Table 4.

TABLE 4
Concentration of WBC and NEC in different batches
of hybridoma cell suspensions in negative urine

| | WBC | NEC |
|---|---|---|
| | concentration | concentration |
| Batch -1 | 37.62/μL | 5.72/μL |
| (500 μL cell suspension in | | |
| 5.0 mL Urine) | | |
| Batch -2 | 76.56/μL | 8.80/μL |
| (500 μL cell suspension in | | |
| 2.5 mL Urine) | | |
| Batch -3 | 94.38/μL | 8.36/μL |
| (500 μL cell suspension in | | |
| 2.5 mL Urine) | | |
| Batch -4 | 92.40/μL | 7.48/μL |
| (500 μL cell suspension in | | |
| 2.5 mL Urine) | | |
| Batch -5 | 90.42/μL | 14.96/μL |
| (500 μL cell suspension in | | |
| 2.5 mL Urine) | | |
| Batch -6 | 73.26/μL | 11.44/μL |
| (500 μL cell suspension in | | |
| 2.5 mL Urine) | | |

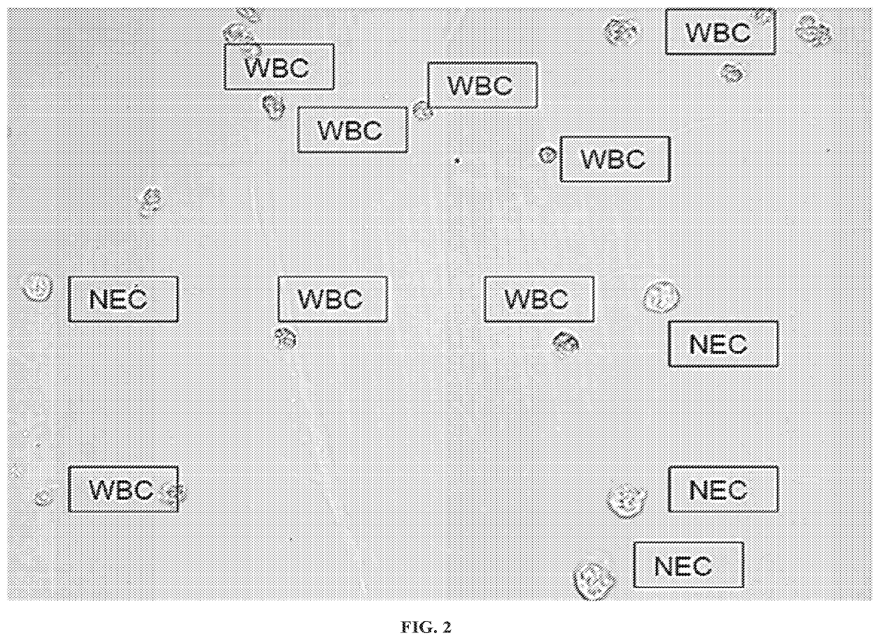

The above results indicate that the hybridoma cells are recognized as WBC and NEC by the Urised2. FIG. 2 is a representative image of WBC and NEC positive samples prepared by adding hybridoma cell suspension in urine.

Preparation of NEC Positive Sample from Human Lung Cancer Epithelial Cells (M1975)

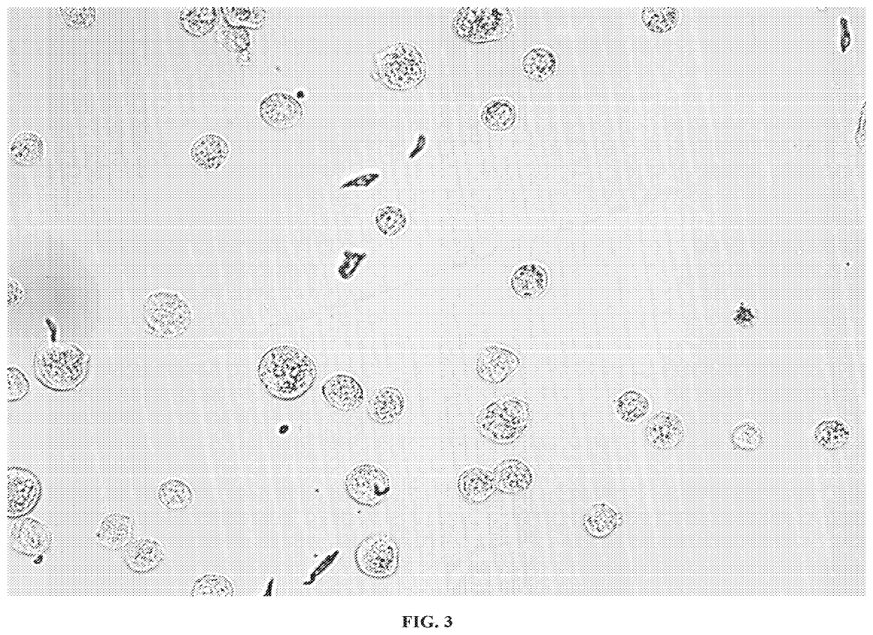

M1975 positive urine was prepared as follows:
1) Human M1975 cells were grown in DMEM medium and were fixed in 2% formaldehyde at 2-8° C. for 24 hr. The preserved cells were stored in PBS (with BSA and Azide) for 24 hr.
2) Cell suspension (100 μL) was added in negative urine (3 mL). A 3 mL aliquot was analyzed on Urised2. The results indicated a NEC concentration=31.68/μL.
3) Cell suspension (500 μL) was added in negative urine (3 mL). A 3 mL aliquot was analyzed on Urised2. The results indicated a NEC concentration=176.44/μL. (FIG. 3)
4) 1.5 mL of 500 μL-suspension was diluted with 1.5 mL negative urine (batch-2 stock). A 3 mL aliquot was analyzed on Urised2. As shown in Table 5, the results indicated a NEC concentration=77.88/μL.
5) To check reproducibility, another set was prepared by adding cell suspension (500 μL) in negative urine (3 mL). A 3 mL aliquot was analyzed on Urised2. The results indicated a NEC concentration=164.12/μL.
6) 1.5 mL of second batch of 500 μL-suspension was diluted with 1.5 mL negative urine (batch-3 stock). A 3 mL aliquot was analyzed on Urised2. As show in Table 5, the results indicated a NEC concentration=82.28/μL.

TABLE 5
Concentration of NEC in different batches of NEC-
positive urine materials prepared by adding M1975
cell suspension added in to negative urine

| | NEC | NEC |
|---|---|---|
| | concentration | concentration |
| | in the stock | in 1:1 diluted stock |
| Batch -1 stock | 31.68/μL | 5.72/μL |
| (100 μL cell | | |
| suspension in | | |
| 3.0 mL Urine) | | |
| Batch -2 stock | 176.44/μL | 77.88/μL |
| (500 μL cell | | |
| suspension in | | |
| 3.8 mL Urine) | | |
| Batch -3 stock | 164.12/μL | 82.28/μL |
| (500 μL cell | | |
| suspension in | | |
| 3.8 mL Urine) | | |

FIG. 3 is a representative Urised2 image of Batch-3 stock, with a NEC concentration of 82.28/μL. The image shows the presence of round shaped cells with defined smooth edges.

The reproducibility of the preparation was tested by separately growing M1975 cells following the above procedure and then adding two different aliquots of cell suspensions (100 μL) in sediment negative urine (5 mL).

TABLE 6
Concentration of NEC in different batches of
NEC-positive urine materials prepared by adding
M1975 cell suspension to negative urine

| | NEC concentration |
|---|---|
| | in the stock |
| Batch-1 stock (100 μL cell | 14.98/μL |
| suspension in 5 mL urine) | |
| Batch -2 stock (100 μL blood | 11.00/μL |
| in 3.8 mL urine) | |

Preparation of YEA Positive Sample from Yeast Cells

Yeast positive urine was prepared as follows:
1) Yeast cells (Penicillium roqueforti or Baker's yeast Saccharomyces cerevisiae) were grown separately in PBS solution and in negative urine in the presence of glucose (approximately 0.5%) at 37° C.
2) Yeast cells, obtained from yeast positive clinical urine specimen, were also grown by adding 1 mL of yeast positive urine sample into 40 mL sediment negative urine. The yeast cells found in clinical urine samples are typically Candida albicans. The yeast cells were grown both in the absence of carbohydrate and in the presence of 1 mL 5% sucrose solution. The admixture was kept at room temperature for 24 hours to allow yeast cells to grow.
3) Yeast cells grown by the above methods were added to negative urine samples and the samples were analyzed in Urised2 and on Atellica UAS 800.

The morphology (round, darker edge) of yeast and red blood cells are very close to each other. Often yeast and red blood cells interfere with each other in microscopy based urinalyses. Similar observations are also found when artificially grown yeast cells are contrived into negative urine sample and analyzed by sediment urinalysis analyzers. Yeast cells grown in the presence of sucrose solution, however, appeared to be detected only as yeast and not falsely detected as red blood cells.

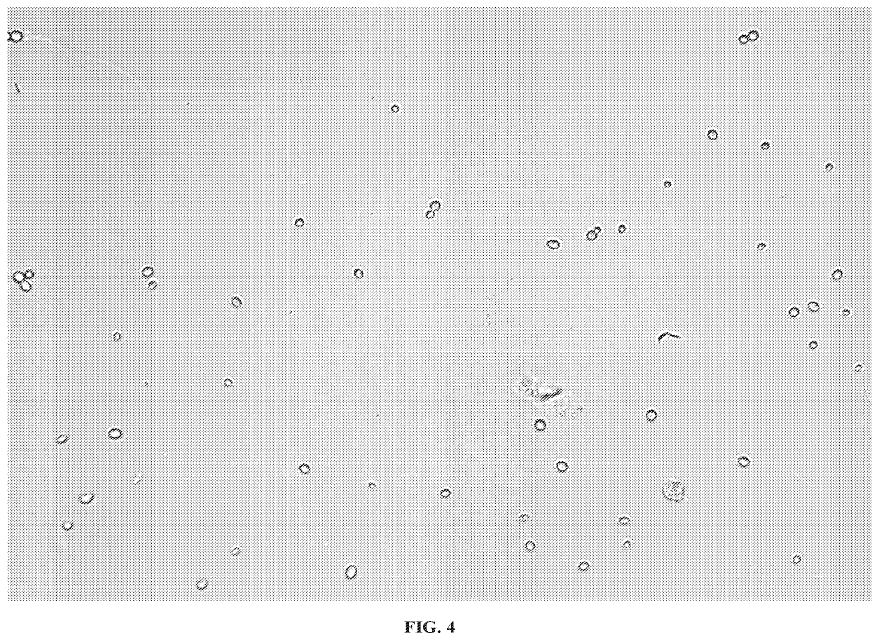

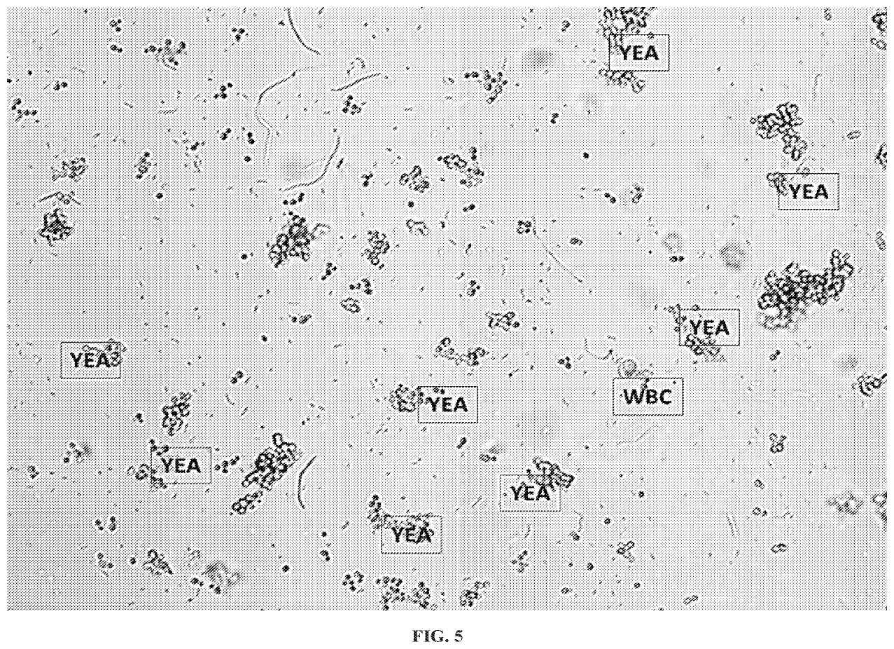

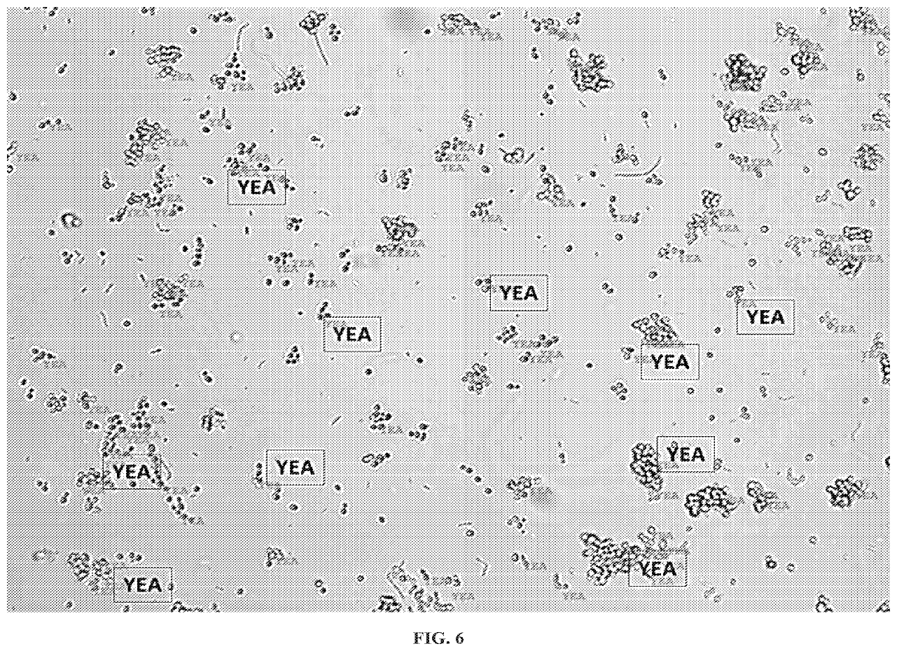

FIG. 4 is a representative Urised2 image of a urine sample (Experiment ID 040616-20 in Table 7), with a YEA concentration of 18.48/μL. The image shows the presence of round-oval shaped single and budding yeast cells. FIG. 5 is a representative Atellica UAS 800 image of a urine sample (Experiment ID=YEA-2 AM-1 in Table 7), with a YEA concentration of 80-100/μL. The image shows the presence of round-oval shaped single and budding yeast cells. FIG. 6 is a representative Atellica UAS 800 image of a urine sample (Experiment ID=11082017-YEA-Contrived in Table 7), with a YEA concentration of 652-674/μL. The image shows the presence of round-oval shaped single and budding yeast cells.

TABLE 7
Concentration of YEA in different batches of YEA-positive
urine materials prepared by adding laboratory grown
yeast cells suspension to negative urine

| | | YEA | RBC |
|---|---|---|---|
| Experiment | | concentration in | concentration in |
| ID | Yeast source | the result | the result |
| 040616-20 | Baker's Yeast | 14.98/μL | 272.80/μL |
| | grown in PBS | | |
| YEA-2AM-1 | Clinical yeast | 12.76/μL | 78.32/μL |
| | grown in negative | | |
| | urine without | | |
| | sucrose | | |
| 11082017- | Clinical yeast | 652/μL | 0.0/μL |
| YEA-Contrived | grown in negative | | |
| | urine in the | | |
| | presence sucrose | | |

Preparation of MUC Positive Sample from Egg White

In general, egg white contains ˜90% water and ˜10% protein as its major components, but also contains a small amount of carbohydrate and sodium salt. The egg white has a thick mucoid appearance. Egg white positive urine was prepared as follows:
1) Approximately 1 mL of egg white was added in 5 mL of negative urine.
2) The admixture was mixed by very slowly inverting the container.
3) The sample was analyzed on Atellica UAS 800.

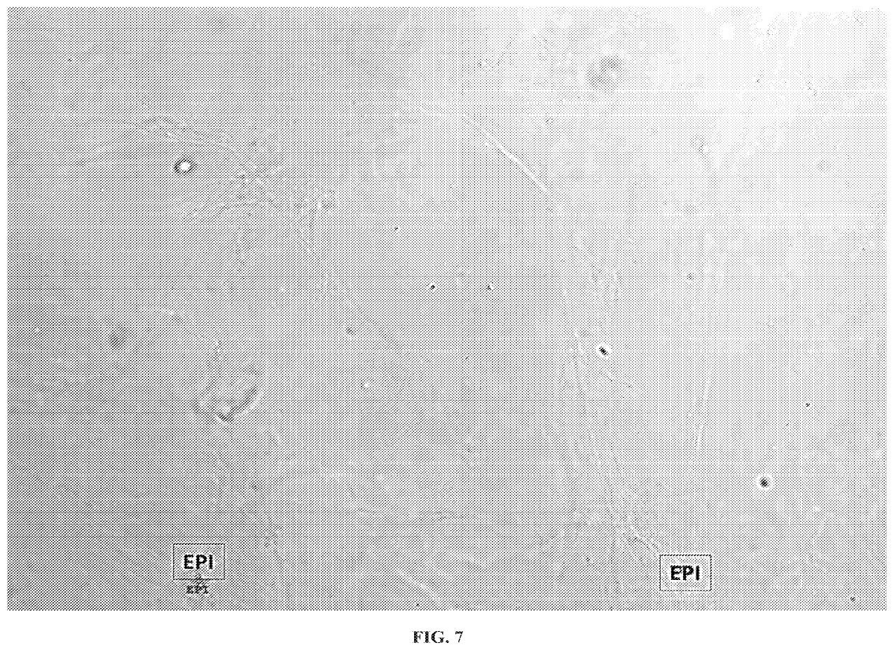

Table 8 and FIG. 7 indicate that adding egg white in negative urine results in positive mucus concentration.

TABLE 8
Concentration of MUC in samples with
egg white added into negative urine

| Experiment ID | MUC concentration |
|---|---|
| Egg White in Negative Urine-1 | 417.6/μL (Positive - based on |
| | 200/μL abnormal threshold) |
| Egg White in Negative Urine + | 322.96/μL (Positive - based on |
| Sodium Chloride-1 | 200/μL abnormal threshold) |

Preparation of Pathological Cast (PAT) Positive Sample from Hydrodictyon Algae

The following procedure was followed to obtain PAT positive urine samples:
1) 10 mL of Hydrodictyon algae suspension in water was centrifuged at 3000 rpm for 2 minutes. After the centrifugation, the supernatants were removed and the residues were resuspended in remaining 1 mL volume suspension.
2) The 1 mL centrifuged resuspension was mixed with 3 mL of negative urine.
3) The mixed samples were tested on Urised2.

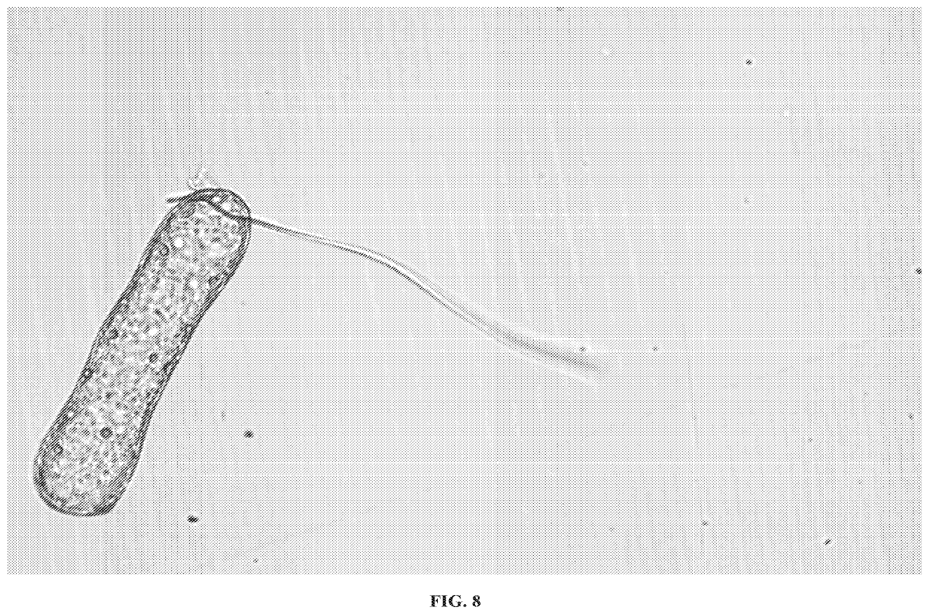

The results indicated the presence of PAT (3.08/μL) as shown in FIG. 8.

As shown in the above examples, in addition to the similarity in the appearances observed in the microscopy images, the corresponding results from the urine sediment analyzer indicated that the instruments were also recognizing these analogues as urine sediment analytes. The disclosed quality control substances therefore enable better evaluation of the performance of sediment analyzers, which measure pathologically important analytes such as PAT, NEC and YEA.

Those skilled in the art will appreciate that numerous changes and modifications can be made to the preferred embodiments of the quality control substances and methods and that such changes and modifications can be made without departing from the spirit of the disclosed subject matter. It is, therefore, intended that the appended claims cover all such equivalent variations as fall within the true spirit and scope of the invention.

### Embodiments

The following list of embodiments is intended to complement, rather than displace or supersede, the previous descriptions.
Embodiment 1. A quality control substance for use with a microscopy-based urine sediment analyzer, the quality control substance comprising:
   a urine matrix; and
   a cancer cell, an algae cell, a yeast cell, egg white, or any combination thereof.
Embodiment 2. The quality control substance of embodiment 1, wherein the cancer cell is an SKBR-3 cell or an H-1975 cell.
Embodiment 3. The quality control substance of embodiment 1, wherein the algae cell is diatom.
Embodiment 4. The quality control substance of embodiment 1, wherein the yeast cell is a naturally occurring yeast (Candida Albican) present in urine or Saccharomyces cerevisiae.
Embodiment 5. The quality control substance of any one of the previous embodiments, further comprising a crystal, a bacterial cell, a sperm cell, a white blood cell, a red blood cell, a hyaline cast, or any combination thereof.
Embodiment 6. A method of detecting the presence of an analyte in a urine sample from a subject comprising:
   analyzing the quality control substance of any one of embodiments 1-5 with a microscopy-based urine sediment analyzer to determine a morphology of components within the quality control substance and
   comparing the morphology of the components within the quality control substance to a morphology of analytes within the urine sample, wherein a matching morphology between the analyte and the quality control substance indicates the presence of the analyte in the urine sample.
Embodiment 7. The method of embodiment 6, wherein:
   (i) the analyte is a non-squamous epithelial cell and the quality control substance comprises a cancer cell;
   (ii) the analyte is a pathological cast and the quality control substance comprises an algae cell;
   (iii) the analyte is a yeast cell and the quality control substance comprises a yeast cell;
   (iv) the analyte is mucus and the quality control substance comprises egg white; or
   (v) any combination of (i)-(iv).
Embodiment 8. The method of embodiment 7, wherein the cancer cell is an SKBR-3 cell or an H-1975 cell.
Embodiment 9. The method of embodiment 7, wherein the algae cell is diatom.
Embodiment 10. The method of embodiment 7, wherein the yeast cell is a naturally occurring yeast (Candida Albican) present in urine or Saccharomyces cerevisiae.
Embodiment 11. The quality control substance of any one of embodiments 1-5 for use in identifying the presence of one or more analytes in a urine sample from a subject.
Embodiment 12. Use of a cancer cell, an algae cell, a yeast cell, egg white, or any combination thereof in the manufacture of a quality control substance for use with a microscopy-based urine analyzer.

1. A quality control substance for use with a microscopy-based urine sediment analyzer, the quality control substance comprising:
a urine matrix; and
a cancer cell, an algae cell, a yeast cell, egg white, or any combination thereof.

2. The quality control substance of claim 1, wherein the cancer cell is an SKBR-3 cell or an H-1975 cell.

3. The quality control substance of claim 1, wherein the algae cell is diatom.

4. The quality control substance of claim 1, wherein the yeast cell is a naturally occurring yeast (Candida Albican) present in urine or Saccharomyces cerevisiae.

5. The quality control substance of claim 1, further comprising a crystal, a bacterial cell, a sperm cell, a white blood cell, a red blood cell, a hyaline cast, or any combination thereof.

6. A method of detecting the presence of an analyte in a urine sample from a subject comprising:
analyzing the quality control substance of claim 1 with a microscopy-based urine sediment analyzer to determine a morphology of components within the quality control substance and
comparing the morphology of the components within the quality control substance to a morphology of analytes within the urine sample, wherein a matching morphology between the analyte and the quality control substance indicates the presence of the analyte in the urine sample.

7. The method of claim 6, wherein:
(i) the analyte is a non-squamous epithelial cell and the quality control substance comprises a cancer cell;
(ii) the analyte is a pathological cast and the quality control substance comprises an algae cell;
(iii) the analyte is a yeast cell and the quality control substance comprises a yeast cell;
(iv) the analyte is mucus and the quality control substance comprises egg white; or
(v) any combination of (i)-(iv).

8. The method of claim 7, wherein the cancer cell is an SKBR-3 cell or an H-1975 cell.

9. The method of claim 7, wherein the algae cell is diatom.

10. The method of claim 7, wherein the yeast cell is a naturally occurring yeast (Candida Albican) present in urine or Saccharomyces cerevisiae.

11. The quality control substance of claim 1 for use in identifying the presence of one or more analytes in a urine sample from a subject.

12. Use of a cancer cell, an algae cell, a yeast cell, egg white, or any combination thereof in the manufacture of a quality control substance for use with a microscopy-based urine analyzer.
